(19) United States
(12) Patent Application Publication
(10) Pub. No.: US 2017/0153217 A1
(43) Pub. Date: Jun. 1, 2017

(54) MICROFLUIDIC WATER SAMPLE ANALYSIS KIT

(71) Applicant: Palintest Limited, Gateshead, Tyne & Wear (GB)

(72) Inventor: Simon JOHNSTON, Chollerton (GB)

(73) Assignee: Palintest Limited, Gateshead, Tyne & Wear (GB)

(21) Appl. No.: 15/322,799

(22) PCT Filed: Jul. 9, 2015
(86) PCT No.: PCT/GB2015/051990

§ 371 Date: Dec. 29, 2016

(30) Foreign Application Priority Data
Aug. 5, 2014 (GB) 1413857.2

(51) Int. Cl. G01N 33/18; B01L 3/00

(52) U.S. Cl. CPC G01N 33/1893; G01N 33/182; B01L 3/502723; B01L 2200/16; B01L 2300/0864; B01L 2300/161; B01L 2400/0688

(57) ABSTRACT

The present invention relates to a water sample analysis kit for determining the presence or amount (eg concentration) of multiple chemical analytes in a water sample.

The present invention relates to a water sample analysis kit for determining the presence or amount (eg concentration) of multiple chemical analytes in a water sample.

Conventional devices for everyday point-of-use measurements of a chemical analyte in water typically rely on pre-preparing suitable reagents. For example, reagent tablets may be placed in a measurement cell and agitated before use. A simpler and more rapid test may be carried out with a test strip but the results are generally less accurate. The measurement of multiple chemical analytes is typically carried out one-by-one and the procedure is laborious.

The present invention seeks to improve the analysis of multiple chemical analytes in a water sample (eg an aliquot of water).

Thus viewed from a first aspect the present invention provides a water sample analysis kit for determining the presence or amount of a first chemical analyte and a second chemical analyte which are different, said water sample analysis kit comprising:

a cartridge in which is fabricated an array of discrete fluidic channels each facilitating a water sample flow from a common entry port, wherein the array of discrete fluidic channels comprises:
a first discrete fluidic channel terminating at a first exit port;
a first substrate or first well in or adjacent to the first discrete fluidic channel;
a first analytical cell in the first discrete fluidic channel downstream from the first substrate or first well to receive a first amount of the water sample, wherein the first substrate, the first well, the first analytical cell and a part of the first discrete fluidic channel adjacently upstream to the first analytical cell are selectively loaded with first reagents whereby in use to expose the water sample flow to the first reagents to cause the formation of a first product from the first chemical analyte and the first reagents;
a second discrete fluidic channel terminating at a second exit port;
a second analytical cell in the second discrete fluidic channel to receive a second amount of the water sample, wherein the second analytical cell or a part of the second discrete fluidic channel adjacently upstream to the second analytical cell is selectively loaded with second reagents whereby in use to expose the water sample flow to the second reagents to cause the formation of a second product from the second chemical analyte and the second reagents,
   wherein the first exit port and second exit port are discrete or common and the first and second discrete fluidic channels are air-vented at or near to the first exit port and second exit port by a hydrophobic air-venting membrane or a hydraulically resistant air-venting discontinuity or dislocation; and
   a detector in which the cartridge is dismountably mounted or mountable so as to enable the detector (1) to detect in the first analytical cell a first measurable response to the occurrence or degree of formation of the first product or to the occurrence or degree of depletion of a first reagent and to relate the first measurable response to the presence or amount of the first chemical analyte and (2) to detect in the second analytical cell a second measurable response to the occurrence or degree of formation of the second product or to the occurrence or degree of depletion of a second reagent and to relate the second measurable response to the presence or amount of the second chemical analyte.

The water sample analysis kit of the invention advantageously permits straightforward and rapid on-site (point-of-use) analysis with minimal reagent preparation. The hydrophobic or hydraulically resistant nature of the air vent ensures sufficiently rapid water sample flow and a consistent amount of water sample in the analytical cell to facilitate accurate quantitative measurements.

Each discrete fluidic channel may be characterised by a dimension (eg diameter) of 1 millimeter or less. Each discrete fluidic channel may be characterised by a sub-millimeter dimension (eg diameter). Each discrete fluidic channel may be a discrete microfluidic channel. For example the diameter of each discrete microfluidic channel may be in the range 100 nm to 500 μm.

Preferably the first exit port and second exit port are common and the first and second discrete fluidic channels are air-vented by a common hydrophobic air-venting membrane.

Particularly preferably the common hydrophobic air-venting membrane is mounted near to the common first exit port and second exit port in a bore (eg a vertical bore) which is fluidly connected to the first and second discrete fluidic channels.

The hydrophobic air-venting membrane may be porous (eg microporous).

The hydrophobic air-venting membrane may have a breakthrough pressure in the range 6 to 80 psi.

The hydrophobic air-venting membrane may be composed of a hydrophobic polymer such as an optionally fluorinated polyurethane, polyalkylene or polyolefin or a silicone. Preferably the hydrophobic membrane is composed of polytetrafluoroethylene (PTFE).

Preferably the first exit port and second exit port are discrete and each of the first and second discrete fluidic channels is air-vented by a hydraulically resistant air-venting discontinuity. Particularly preferably the hydraulically resistant air-venting discontinuity is a constriction in a neck of each of the first and second discrete fluidic channels near to the first and second exit ports (eg downstream from the analytical cell). Typically the constriction has a diameter of about 500 μm. The length of the constriction may be adjusted for pressure control.

The first exit port and second exit port may be discrete and each of the first and second discrete fluidic channels may be air-vented by a hydraulically resistant air-venting dislocation.

In the cartridge may be fabricated a collection reservoir downstream from the array of discrete fluidic channels. The collection reservoir serves to collect any water sample which exits the first exit port and second exit port.

The first measurable response and second measurable response may be measurable electrochemically or spectrophotometrically.

Preferably the first measurable response and second measurable response are measured spectrophotometrically (eg colourimetrically or fluorimetrically).

The first measurable response and second measurable response may be attenuation or development of colour, absorbance, transmittance or reflectance (eg UV, Vis or IR absorbance, transmittance or reflectance).

Preferably the first measurable response and second measurable response is attenuation or development of colour.

Preferably the detector is enabled to detect substantially simultaneously the first measurable response and the second measurable response.

Preferably the detector is enabled to relate substantially simultaneously the first measurable response to the presence or amount of the first chemical analyte and the second measurable response to the presence or amount of the second chemical analyte.

The detector may be an array of individual detector elements alignable with the analytical cells in the cartridge for illumination and detection. The individual detector elements may address the analytical cells substantially simultaneously.

The detector may be enabled to relate the first measurable response to the presence or amount of the first chemical analyte and the second measurable response to the presence or amount of the second chemical analyte by conventional methodologies and processing familiar to those skilled in the art and the detector may be programmed accordingly.

The first substrate or first well may be loaded with first reagents which in use are flushed into the water sample flow and carried downstream to the first analytical cell.

Preferably the first analytical cell is loaded with first reagents (eg in a first reagent-loaded substrate) located in the first analytical cell.

Preferably the second analytical cell is loaded with second reagents in a second reagent-loaded substrate located in the second analytical cell.

A reagent-loaded substrate advantageously exposes a large surface area of dispersed reagent to the water sample flow which leads to rapid dissolution and/or reaction.

Typically each substrate is a high surface area substrate. Each substrate is typically inert. Each substrate may be porous, fibrous or reticulated. Each substrate may be paper, fabric, web or mesh. Suitable substrates are generally well-known and commercially available from (for example) Whatman, Saati, Sericol or Ahlstrom.

Each substrate or well may be loaded with a deposited, dried, dosed (eg microdosed) or printed reagent.

The reagent may be a solid reagent which on exposure to the water sample is dissolved, dispersed or suspended. For example, the reagent may be a dehydrated reagent which is rehydratable on exposure to the water sample.

The reagent may be soluble or at least partially (eg fully) insoluble.

The reagent may be a buffer, oxidant, reductant, acid or alkali.

The first and second reagents suitable for testing first and second chemical analytes respectively will be familiar to those skilled in the art and are commercially available.

The water sample may be introduced to the cartridge by dipping or dosing.

The water sample flow may be passive (eg capillary) flow or active (eg differential pressure) flow. Differential pressure flow may be generated by a pump or vacuum.

The water sample analysis kit may further comprise a flow actuator detachably attached to the common entry port for actuating water sample flow along the array of discrete fluidic channels. The flow actuator may be a syringe, micropump or microvalve.

In a preferred embodiment, the array of discrete fluidic channels further comprises:
one or more additional discrete fluidic channels each of which terminates at an additional exit port;
an optional additional substrate or additional well in or adjacent to each additional discrete fluidic channel;
an additional analytical cell downstream from the optional additional substrate or additional well in each additional discrete fluidic channel to receive an additional amount of the water sample, wherein one or more of the optional additional substrate, the additional well, the additional analytical cell and a part of the additional discrete fluidic channel adjacently upstream to the additional analytical cell are loaded with additional reagents whereby in use to expose the water sample flow to the additional reagents to cause the formation of an additional product from an additional chemical analyte and the additional reagents.

The number of additional discrete fluidic channels and associated additional features may be one or more (eg five).

The characteristics of the additional discrete fluidic channels, additional exit port, additional substrate, additional well, additional analytical cell, additional amount of water sample, additional product, additional chemical analyte and additional reagents may be the same as or different from the characteristics of the corresponding first and second such features described hereinbefore.

In a preferred embodiment, the array of discrete fluidic channels further comprises:
a non-loaded discrete fluidic channel which terminates at an exit port;
an optional non-loaded substrate or non-loaded well in or adjacent to the non-loaded discrete fluidic channel;
a non-loaded analytical cell downstream from the optional non-loaded substrate or non-loaded well in the non-loaded discrete fluidic channel.

The non-loaded analytical cell may be used for referencing or correction purposes or for use in feedback and validation or as a location identifier.

Each substrate or well in each discrete fluidic channel may be one of a plurality of sequential substrates or wells. The plurality of sequential substrates or wells may be in series or parallel. The depth of the plurality of wells may vary.

The array of discrete fluidic channels may further comprise a particulate filter mounted in a discrete fluidic channel.

The array of discrete fluidic channels may be an array of discrete non-linear fluidic channels. The array of discrete fluidic channels may be an array of discrete non-tortuous fluidic channels.

Preferably the array of discrete fluidic channels is an array of discrete angular (eg zig-zag) fluidic channels. Particularly preferably each of the array of discrete fluidic channels is angular downstream from the analytical cell.

The discrete fluidic channel may be constricted (eg upstream from the analytical cell). The discrete fluidic channel may be branched.

The cartridge may be polymeric (eg composed of polyester, polycarbonate or polyvinyl chloride). The cartridge may be in the form of a card or sheet. The cartridge may be a separable one of a plurality of cartridges in a continuous form (eg a roll). The cartridge may be single-use.

The cartridge may be configured to introduce turbulence to the water sample flow.

The cartridge may comprise an upper transparent body and a lower opaque body. The upper transparent body and lower opaque body may be ultrasonically bonded or mechanically fastened. The upper transparent body may be equipped with a connection for a syringe (eg a Luer connector). The upper transparent body and a lower opaque body may be moulded.

The first exit port and second exit port may be discrete and each of the first and second discrete fluidic channels is air-vented by a hydraulically resistant air-venting dislocation. For example, the upper transparent body and lower opaque body may be fastened in a cantilevered fashion and the cantilever operates under sufficient air pressure to dislocate the upper transparent body and lower opaque body to release air but remain hydraulically resistant.

The common entry port may be in an edge or an upper face or lower face of the cartridge. The common entry port may be an injection port.

The cartridge may be mounted or mountable in the detector by a conventional mechanical connection such as a cantilever or cam. The cartridge may be substantially planar with a convenient polygonal profile. The cartridge may be equipped with peripheral features to assist mounting and dismounting from the detector. The cartridge may be equipped with a grip to assist mounting and dismounting from the detector. This helps to prevent fouling of the optical surfaces.

Preferably the first and second (and any additional) chemical analytes is selected from the group consisting of phosphate, chlorine dioxide, free chlorine, chlorite, cyanuric acid, total chlorine, calcium or magnesium.

Preferably the amount of the first and second (and any additional) chemical analytes is the concentration, pH or alkalinity.

Typically the water sample is an aqueous sample (eg an aqueous solution, suspension or dispersion). The water sample may be a sample of potable water, recreational water (eg swimming pool water), environmental water or waste water (eg industrial waste water). The water sample may be chlorine dioxide-containing.

The volume of the water sample may be a μL, nL, pL or fL volume.

The cartridge or water sample analysis kit may be portable. The water sample analysis kit may be an on-site (point-of-use) water sample analysis kit.

Viewed from a further aspect the present invention provides a cartridge as hereinbefore defined.

The present invention will now be described in a non-limitative sense with reference to the accompanying drawings in which:

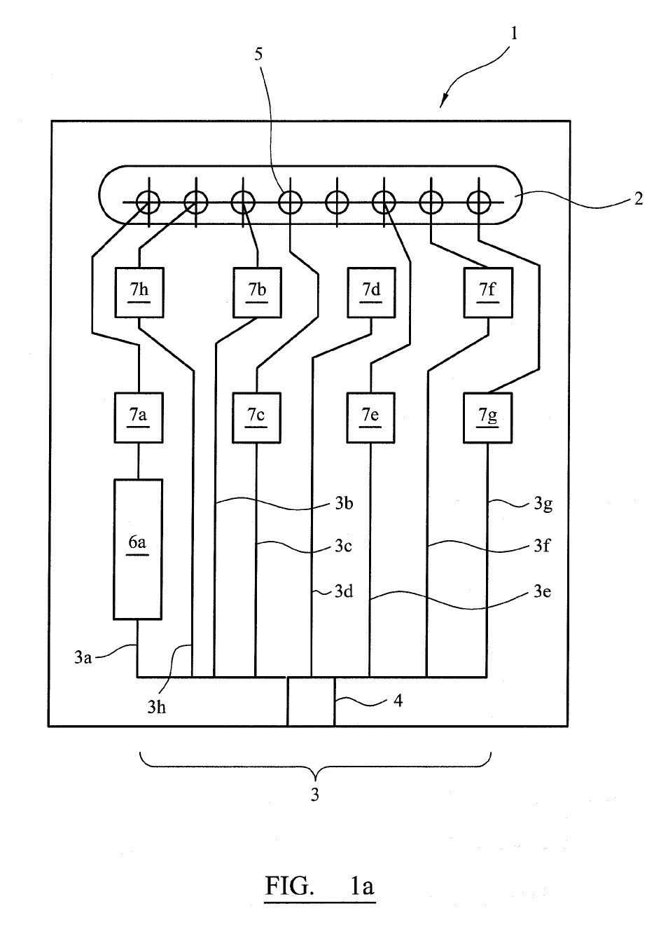

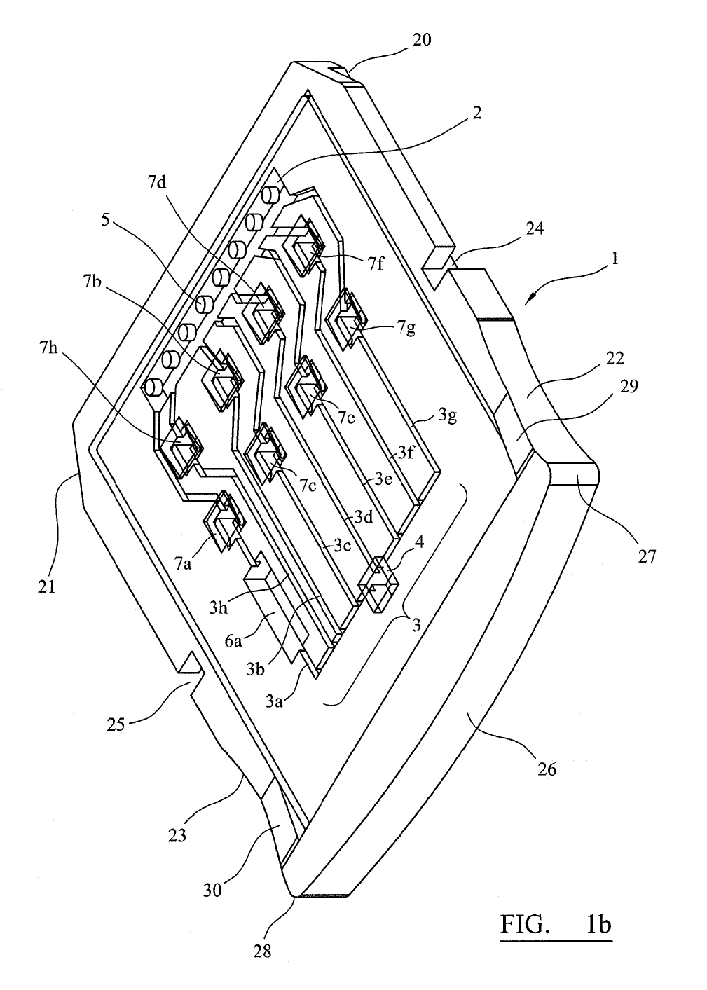

FIG. 1a illustrates a schematic plan view of a cartridge of an embodiment of the water sample analysis kit of the present invention;

FIG. 1b illustrates a detailed schematic perspective view of the cartridge of the embodiment of the water sample analysis kit of the present invention;

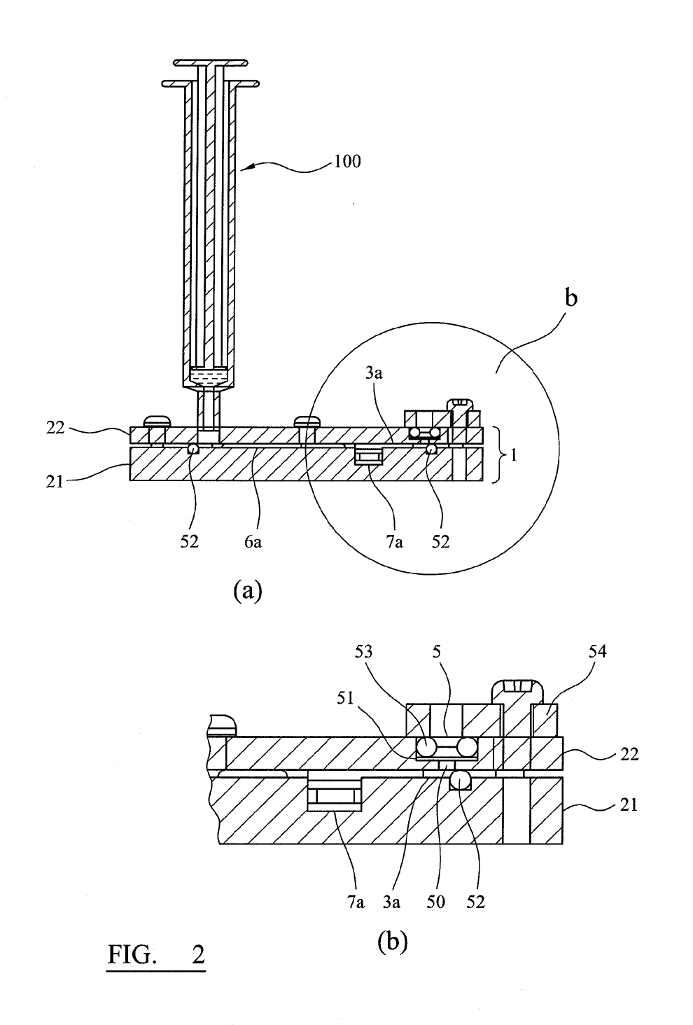

FIG. 2 illustrates (a) a cross-sectional view through a discrete fluidic channel of the cartridge of the embodiment of the water sample analysis kit of the present invention and (b) a close-up view of part b shown in 2(a); and

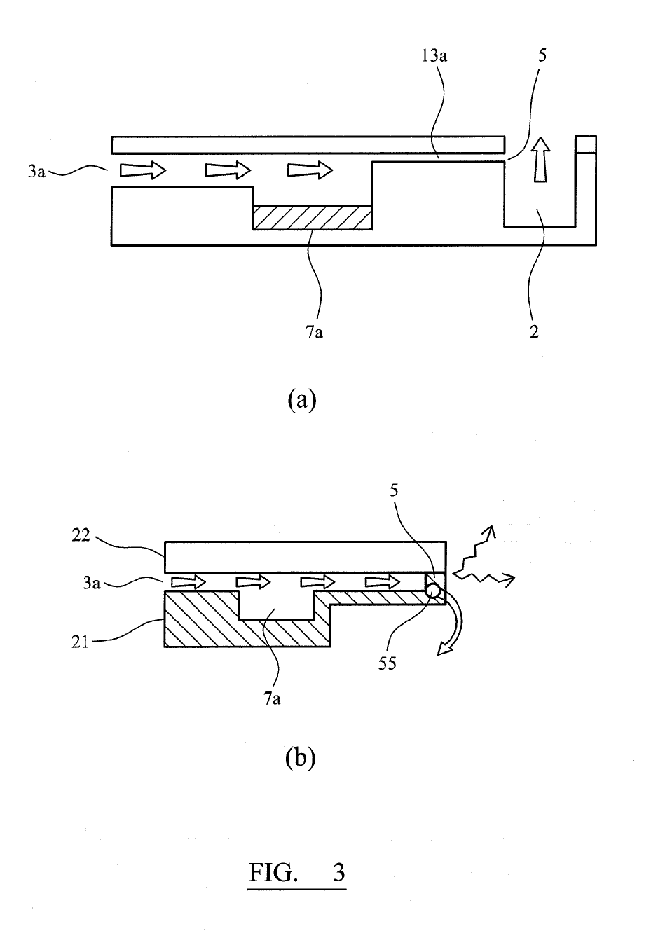

FIGS. 3(a) and (b) illustrate schematic cross-sectional views through alternative exit ports of a discrete fluidic channel of a cartridge of an embodiment of the water sample analysis kit of the present invention.

FIGS. 1a and 1b illustrate schematic views of a cartridge of an embodiment of the water sample analysis kit of the present invention designated generally by reference numeral 1. In the cartridge 1 is fabricated an array of discrete fluidic channels 3 each permitting a water sample flow from a common entry port 4 to an exit port 5. Each of the array of discrete fluidic channels 3 is angular. The exit port 5 is described in more detail below with reference to FIG. 2. In the cartridge 1 is also fabricated a collection reservoir 2 downstream from the array of discrete fluidic channels 3 which serves to collect water which escapes the exit ports 5. The cartridge 1 is mountable in and dismountable from a detector (not shown).

The array of discrete fluidic channels 3 comprises a first discrete fluidic channel 3a. In the first discrete fluidic channel 3a is a first reagent-loaded substrate 6a loaded with reagents for phosphate ions and a first analytical cell 7a downstream from the first reagent-loaded substrate 6a to receive a first amount of the water sample. There is a chemical change amongst the phosphate ion present in the water sample and the reagents for phosphate. A device such as a TAOS detector can be used to make a spectrophotometric measurement of the water sample in the first analytical cell 7a which can be related to the presence or amount (eg concentration) of phosphate ion in the water sample.

The array of discrete fluidic channels 3 further comprises a second discrete fluidic channel 3b. In the second discrete fluidic channel 3b is a second analytical cell 7b to receive a second amount of the water sample. The second analytical cell 7b is loaded with reagents for cyanuric acid. There is a chemical change amongst the cyanuric acid present in the water sample and the reagents for cyanuric acid. The TAOS detector can be used to make a spectrophotometric measurement of the water sample in the second analytical cell 7b which can be related to the presence or amount (eg concentration) of cyanuric acid in the water sample.

The array of discrete fluidic channels 3 further comprises a third discrete fluidic channel 3c. In the third discrete fluidic channel 3c is a third analytical cell 7c to receive a third amount of the water sample. The third analytical cell 7c is loaded with reagents for alkalinity. There is a chemical change in the reagents for alkalinity. The TAOS detector can be used to make a spectrophotometric measurement of the water sample in the third analytical cell 7c which can be related to the alkalinity in the water sample.

The array of discrete fluidic channels 3 further comprises a fourth discrete fluidic channel 3d. In the fourth discrete fluidic channel 3d is a fourth analytical cell 7d to receive a fourth amount of the water sample. The fourth analytical cell 7d is loaded with reagents for total chlorine. There is a chemical change amongst the total chlorine present in the water sample and the reagents for total chlorine. The TAOS detector can be used to make a spectrophotometric measurement of the water sample in the fourth analytical cell 7d which can be related to the presence or amount (eg concentration) of total chlorine in the water sample.

The array of discrete fluidic channels 3 further comprises a fifth discrete fluidic channel 3e. In the fifth discrete fluidic channel 3e is a fifth analytical cell 7e to receive a fifth amount of the water sample. The fifth analytical cell 7e is loaded with reagents for free chlorine. There is a chemical change amongst the free chlorine present in the water sample and the reagents for free chlorine. The TAOS detector can be used to make a spectrophotometric measurement of the water sample in the fifth analytical cell 7e which can be related to the presence or amount (eg concentration) of free chlorine in the water sample.

The array of discrete fluidic channels 3 further comprises a sixth discrete fluidic channel 3f. In the sixth discrete fluidic channel 3f is a sixth analytical cell 7f to receive a sixth amount of the water sample. The sixth analytical cell 7f is loaded with reagents for calcium. There is a chemical change amongst the calcium present in the water sample and the reagents for calcium. The TAOS detector can be used to make a spectrophotometric measurement of the water sample in the sixth analytical cell 7f which can be related to the presence or amount (eg concentration) of calcium (eg calcium hardness) in the water sample.

The array of discrete fluidic channels 3 further comprises a seventh discrete fluidic channel 3g. In the seventh discrete fluidic channel 3g is a seventh analytical cell 7g to receive a seventh amount of the water sample. The seventh analytical cell 7g is loaded with reagents for pH. There is a chemical change in the reagents for pH. The TAOS detector can be used to make a spectrophotometric measurement of the water sample in the seventh analytical cell 7g which can be related to the pH of the water sample.

The array of discrete fluidic channels 3 further comprises an eighth discrete fluidic channel 3h. In the seventh discrete fluidic channel 3h is an eighth analytical cell 7h which is non-loaded with reagents (ie is blank). The non-loaded analytical cell 7h may be used for referencing or correction purposes or for use in feedback and validation or as a location identifier.

FIG. 1b shows in more detail the peripheral features which enable the cartridge 1 to be easily mounted in and dismounted from a detector. These features include cut-away corners 20 and 21, shaped edges 22 and 23, slots 24 and 25, a rounded end 26, chamfered corners 27 and 28 and tapered portions 29 and 30 in the upper face.

The various reagents referred to above are commercially available and examples are given in the table below.

| Test | Preferred Formulation |
|---|---|
| Free chlorine | Syringaldazine indicator |
| Total chlorine | Tetramethylbenzidine indicator plus potassium |
| | iodide |
| pH | Phenol Red indicator |
| Alkalinity | Bromocresol Green indicator plus adipic acid |
| Calcium Hardness | o-Cresolphthlalein indicator |
| Cyanuric acid | Melamine plus Thymol Blue and Cresol Red |
| | indicators |
| Phosphate | Molybdenum Blue |

FIG. 2 illustrates (a) a cross-sectional view through the discrete fluidic channel 3a of the embodiment of the water sample analysis kit of the present invention and (b) a close-up view of part b shown in 2(a). From FIG. 2(a), it is apparent that cartridge 1 has an upper transparent moulded body 22 and a lower opaque moulded body 21 which are ultrasonically bonded. The discrete fluidic channel 3a is bound by a continuous seal 52 mounted in the lower opaque moulded body 21 and sealingly engaged with the upper transparent moulded body 22 upon fastening. The upper transparent moulded body 22 is equipped with a Luer connector for a syringe 100.

Near to exit port 5 is a vertical bore 50 fluidly connected to the discrete fluidic channel 3a and passing through upper transparent moulded body 22. Across an intermediate section of the vertical bore 50 is a PTFE membrane 51. The PTFE membrane 51 is sealingly retained across vertical bore 50 by an o-ring 53 and retaining clamp 54.

FIGS. 3(a) and (b) illustrate schematic cross-sectional views through alternative exit ports 5 of a discrete fluidic channel 3a of a cartridge of an embodiment of the water sample analysis kit of the present invention.

In the first alternative, the discrete fluidic channel 3a is equipped with a narrow constriction 13a at the neck of the discrete fluidic channel 3a near to the exit port 5a. The narrow constriction 13a is hydraulically resistant whilst serving to vent air.

In the second alternative, the upper transparent moulded body 22 and lower opaque moulded body 21 are fastened in a cantilevered fashion and separated normally by a seal 55. The cantilever operates under sufficient pressure to dislocate the upper transparent moulded body 22 and lower opaque moulded body 21 to release air but remain hydraulically resistant.

1. A water sample analysis kit for determining the presence or amount of a first chemical analyte and a second chemical analyte which are different, said water sample analysis kit comprising:
a cartridge in which is fabricated an array of discrete fluidic channels each facilitating a water sample flow from a common entry port, wherein the array of discrete fluidic channels comprises:
a first discrete fluidic channel terminating at a first exit port;
a first substrate or first well in or adjacent to the first discrete fluidic channel;
a first analytical cell in the first discrete fluidic channel downstream from the first substrate or first well to receive a first amount of the water sample, wherein the first substrate, the first well, the first analytical cell and a part of the first discrete fluidic channel adjacently upstream to the first analytical cell are selectively loaded with first reagents whereby in use to expose the water sample flow to the first reagents to cause the formation of a first product from the first chemical analyte and the first reagents;
a second discrete fluidic channel terminating at a second exit port;
a second analytical cell in the second discrete fluidic channel to receive a second amount of the water sample, wherein the second analytical cell or a part of the second discrete fluidic channel adjacently upstream to the second analytical cell is selectively loaded with second reagents whereby in use to expose the water sample flow to the second reagents to cause the formation of a second product from the second chemical analyte and the second reagents,
wherein the first exit port and second exit port are discrete or common and the first and second discrete fluidic channels are air-vented at or near to the first exit port and second exit port by a hydrophobic air-venting membrane or a hydraulically resistant air-venting discontinuity or dislocation; and
a detector in which the cartridge is dismountably mounted or mountable so as to enable the detector (1) to detect in the first analytical cell a first measurable response to the occurrence or degree of formation of the first product or to the occurrence or degree of depletion of a first reagent and to relate the first measurable response to the presence or amount of the first chemical analyte and (2) to detect in the second analytical cell a second measurable response to the occurrence or degree of formation of the second product or to the occurrence or degree of depletion of a second reagent and to relate the second measurable response to the presence or amount of the second chemical analyte.

2. A water sample analysis kit as claimed in claim 1 wherein the first exit port and second exit port are common and the first and second discrete fluidic channels are air-vented by a common hydrophobic air-venting membrane.

3. A water sample analysis kit as claimed in claim 2 wherein the common hydrophobic air-venting membrane is mounted near to the common first exit port and second exit port in a bore which is fluidly connected to the first and second discrete fluidic channels.

4. A water sample analysis kit as claimed in claim 1 wherein the first exit port and second exit port are discrete and each of the first and second discrete fluidic channels is air-vented by a hydraulically resistant air-venting discontinuity.

5. A water sample analysis kit as claimed in claim 4 wherein the hydraulically resistant air-venting discontinuity is a constriction in a neck of each of the first and second discrete fluidic channels near to the first and second exit ports.

6. A water sample analysis kit as claimed in claim 1, wherein the detector is enabled to detect substantially simultaneously the first measurable response and the second measurable response.

7. A water sample analysis kit as claimed in claim 1, wherein the detector is enabled to relate substantially simultaneously the first measurable response to the presence or amount of the first chemical analyte and the second measurable response to the presence or amount of the second chemical analyte.

8. A water sample analysis kit as claimed in claim 1, wherein the first analytical cell is loaded with first reagents in a first reagent-loaded substrate located in the first analytical cell.

9. A water sample analysis kit as claimed in claim 1, wherein the second analytical cell is loaded with second reagents in a second reagent-loaded substrate located in the second analytical cell.

10. A water sample analysis kit as claimed in claim 1, wherein the array of discrete fluidic channels further comprises:
one or more additional discrete fluidic channels each of which terminates at an additional exit port;
an optional additional substrate or additional well in or adjacent to each additional discrete fluidic channel;
an additional analytical cell downstream from the optional additional substrate or additional well in each additional discrete fluidic channel to receive an additional amount of the water sample, wherein one or more of the optional additional substrate, the additional well, the additional analytical cell and a part of the additional discrete fluidic channel adjacently upstream to the additional analytical cell are loaded with additional reagents whereby in use to expose the water sample flow to the additional reagents to cause the formation of an additional product from an additional chemical analyte and the additional reagents.

11. A water sample analysis kit as claimed in claim 1, wherein the array of discrete fluidic channels further comprises:
a non-loaded discrete fluidic channel which terminates at an exit port;
an optional non-loaded substrate or non-loaded well in or adjacent to the non-loaded discrete fluidic channel;
a non-loaded analytical cell downstream from the optional non-loaded substrate or non-loaded well in the non-loaded discrete fluidic channel.

12. A water sample analysis kit as claimed in claim 1, wherein the array of discrete fluidic channels is an array of discrete angular fluidic channels.

13. A water sample analysis kit as claimed in claim 12 wherein each of the array of discrete fluidic channels is angular downstream from the analytical cell.

14. A water sample analysis kit as claimed in claim 1, wherein the first and second (and any additional) chemical analytes is selected from the group consisting of phosphate, chlorine dioxide, free chlorine, chlorite, cyanuric acid, total chlorine, calcium or magnesium.

15. A water sample analysis kit as claimed in claim 1, wherein the amount of the first and second (and any additional) chemical analytes is the concentration, pH or alkalinity.

16. (canceled)

17. A cartridge in which is fabricated an array of discrete fluidic channels each facilitating a water sample flow from a common entry port, wherein the array of discrete fluidic channels comprises:
a first discrete fluidic channel terminating at a first exit port;
a first substrate or first well in or adjacent to the first discrete fluidic channel;
a first analytical cell in the first discrete fluidic channel downstream from the first substrate or first well to receive a first amount of the water sample, wherein the first substrate, the first well, the first analytical cell and a part of the first discrete fluidic channel adjacently upstream to the first analytical cell are selectively loaded with first reagents whereby in use to expose the water sample flow to the first reagents to cause the formation of a first product from the first chemical analyte and the first reagents;
a second discrete fluidic channel terminating at a second exit port;
a second analytical cell in the second discrete fluidic channel to receive a second amount of the water sample, wherein the second analytical cell or a part of the second discrete fluidic channel adjacently upstream to the second analytical cell is selectively loaded with second reagents whereby in use to expose the water sample flow to the second reagents to cause the formation of a second product from the second chemical analyte and the second reagents,
wherein the first exit port and second exit port are discrete or common and the first and second discrete fluidic channels are air-vented at or near to the first exit port and second exit port by a hydrophobic air-venting membrane or a hydraulically resistant air-venting discontinuity or dislocation; and
a detector in which the cartridge is dismountably mounted or mountable so as to enable the detector to detect in the first analytical cell a first measurable response to the occurrence or degree of formation of the first product or to the occurrence or degree of depletion of a first reagent and to relate the first measurable response to the presence or amount of the first chemical analyte and to detect in the second analytical cell a second measurable response to the occurrence or degree of formation of the second product or to the occurrence or degree of depletion of a second reagent and to relate the second measurable response to the presence or amount of the second chemical analyte.
